(19) United States
(12) Patent Application Publication
(10) Pub. No.: US 2022/0226082 A1
(43) Pub. Date: Jul. 21, 2022

(54) Dissolving Gingival Retraction Cord Apparatus

(71) Applicant: Erin Wood, Akron, OH (US)

(72) Inventor: Erin Wood, Akron, OH (US)

(21) Appl. No.: 17/152,014

(22) Filed: Jan. 19, 2021

(51) Int. Cl. A61C 9/00; A61K 6/73

(52) U.S. Cl. CPC A61C 9/0033; A61C 2202/00; A61K 6/73

(57) ABSTRACT

A dissolving gingival retraction cord apparatus for minimizing gum irritation includes a container body having a container bottom side, a container sidewall, and an open container top side defining a container inside. A gel solution is coupled within the container body. A lid is coupled to the container body with a dispense aperture extending therethrough. A spool is coupled to the lid and is submerged within the gel solution when the lid is engaged with the container body. A retraction cord is windingly coupled around the spool and has a cord distal end extending through the dispense aperture of the lid. The retraction cord absorbs, or is coated with, the gel solution. The retraction cord is dissolvable after installation within a person's gum tissue.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to dental crown installation devices and more particularly pertains to a new dental crown installation device for minimizing gum irritation. The present device includes a dissolvable retraction cord. The device may also include a dispenser container that impregnates the dissolvable retraction cord with gel.

### (2) Description of Related Art Including Information Disclosed Under 37 CFR 1.97 and 1.98

The prior art relates to dental crown installation devices. Known devices include retractions cords that are not dissolvable and must be dug out after the installation of the crown. This process can cause gingival irritation and bleeding. Other existing devices include dissolvable stents to treat a periodontal defect, yet do not assist in crown installation.

### (h) BRIEF SUMMARY OF THE INVENTION

An embodiment of the disclosure meets the needs presented above by generally comprising a container body having a container bottom side, a container sidewall, and an open container top side defining a container inside. A gel solution is coupled within the container body. A lid is coupled to the container body. The lid has a lid sidewall selectively engageable with the open container top side and a lid top side. The lid top side has a dispense aperture extending from an outer face through an inner face thereof. A spool is coupled to the lid. The spool is coupled to the inner face of the lid top side and is submerged within the gel solution when the lid is engaged with the container body. A retraction cord is coupled to the spool. The retraction cord is windingly coupled around the spool and has a cord distal end extending through the dispense aperture of the lid. The retraction cord absorbs, or is coated with, the gel solution. The retraction cord is dissolvable after installation within a person's gum tissue.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a dissolving gingival retraction cord apparatus according to an embodiment of the disclosure.

FIG. 2 is a side elevation view of an embodiment of the disclosure.

FIG. 3 is an in-use view of an embodiment of the disclosure.

FIG. 4 is a block diagram view of a method of use of an embodiment of the disclosure.

### (j) DETAILED DESCRIPTION OF THE INVENTION

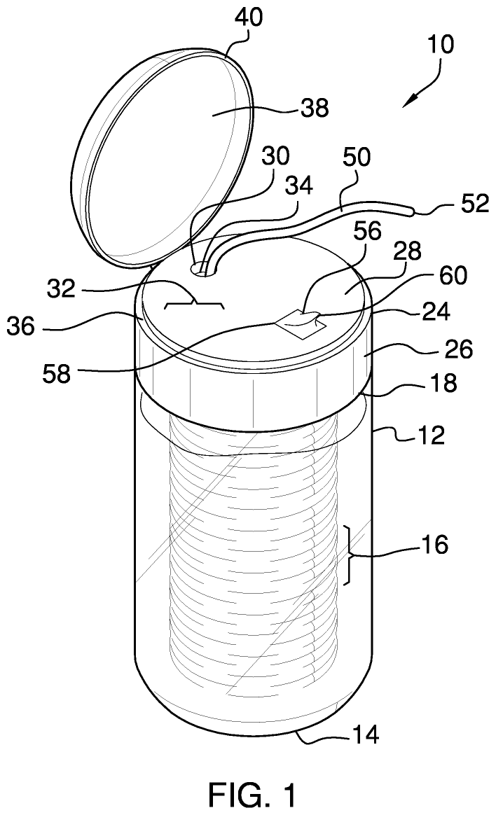

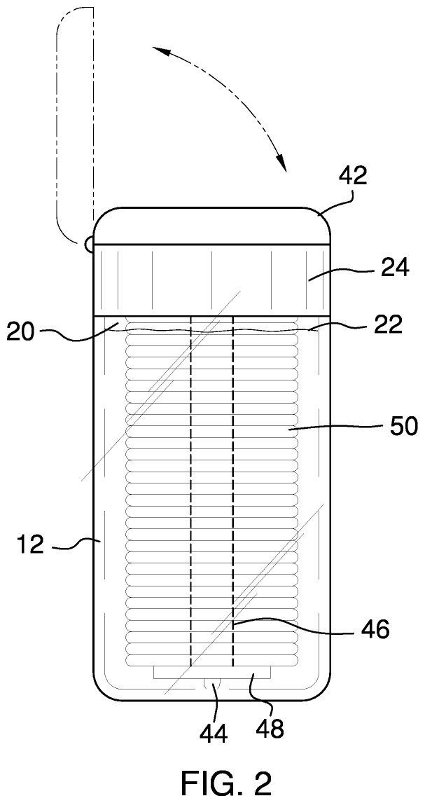

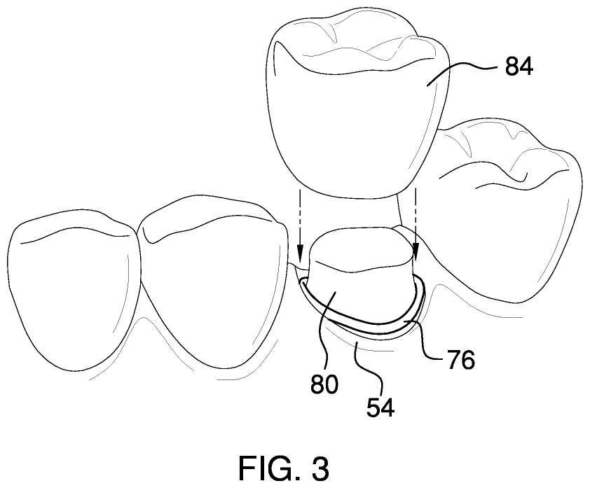

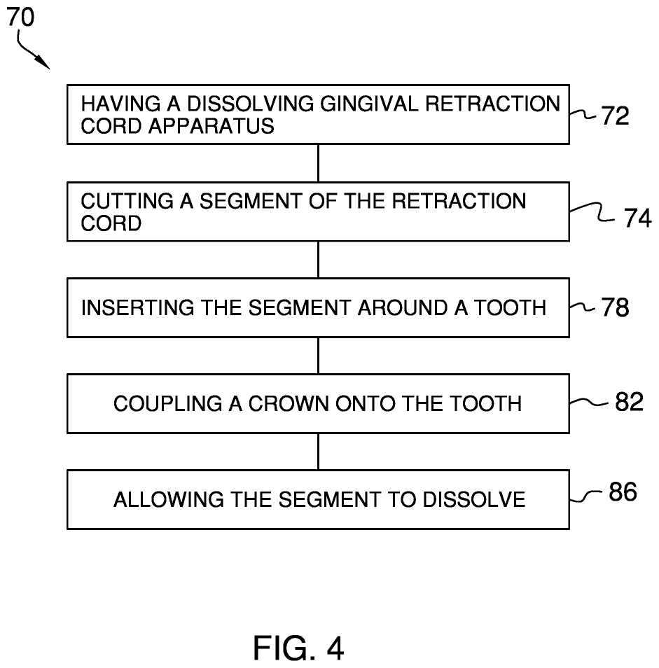

With reference now to the drawings, and in particular to FIGS. 1 through 4 thereof, a new dental crown installation device embodying the principles and concepts of an embodiment of the disclosure and generally designated by the reference numeral 10 will be described.

As best illustrated in FIGS. 1 through 4, the dissolving gingival retraction cord apparatus 10 generally comprises a container body 12 having a container bottom side 14, a container sidewall 16, and an open container top side 18 defining a container inside 20. The container body 12 may be cylindrical. A gel solution 22 is coupled within the container body 12. The gel solution 22 may comprise at least 20% ferric sulfate in order to minimize irritation and bleeding.

A lid 24 is coupled to the container body 12. The lid 24 has a lid sidewall 26 selectively engageable with the open container top side 18 and a lid top side 28. The lid 24 may be press-fit or threaded to the container body 12. The lid top side 28 has a dispense aperture 30 extending from an outer face 32 through an inner face 34 thereof. The lid top side 28 may have a recessed perimeter portion 36.

A cap 38 is coupled to the lid 24. The cap 38 is hingingly coupled to the lid sidewall 26 to selectively cover or expose the lid top side 28. A rim 40 of the cap may selectively engage the recessed perimeter portion 36 of the lid top side to maintain the cap 38 in a closed position covering the lid top side 28 until pulled open. The cap 38 may have a filleted outer perimeter 42.

A spool 44 is coupled to the lid 24. The spool 44 is coupled to the inner face 34 of the lid top side and submerges within the gel solution 22 when the lid 24 is engaged with the container body 12. The spool 44 may include a spool shaft 46 and a spool end cap 48 extending from the spool shaft 46.

A retraction cord 50 is coupled to the spool 44. The retraction cord 50 is windingly coupled around the spool shaft 46 and held in place with the spool end cap 48. The retraction cord 50 has cord distal end 52 extending through the dispense aperture 30 of the lid. The retraction cord 50 absorbs, or is coated with, the gel solution 22. The retraction cord 50 is dissolvable after installation within a person's gum tissue 54. A cutter 56 may be coupled to the lid 24. The cutter 56 is coupled to the lid top side 28 to cut the retraction cord 50. The cutter 56 may include a base portion 58 and a lip portion 60. The lip portion 60 may be shaped to allow the retraction cord 50 to be cut without exposing any portion that could cut the user's finger.

In use, the dissolving gingival retraction cord apparatus 10 allows for a method of use 70 comprising a step 72 comprising having the dissolving gingival retraction cord apparatus 10. A step 74 comprises cutting a segment 76 of the retraction cord 50. A step 78 comprises inserting the segment 76 around a tooth 80 into the gingival sulcus. A step 82 comprises coupling a crown 84 onto the tooth 80. A step 86 comprises allowing the segment 76 to dissolve.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A dissolving gingival retraction cord apparatus comprising:
a container body having a container bottom side, a container sidewall, and an open container top side defining a container inside;
a gel solution coupled within the container body;
a lid coupled to the container body, the lid having a lid sidewall selectively engageable with the open container top side and a lid top side, the lid top side having a dispense aperture extending from an outer face through an inner face thereof;
a spool coupled to the lid, the spool being coupled to the inner face of the lid top side and being submerged within the gel solution when the lid is engaged with the container body; and
a retraction cord coupled to the spool, the retraction cord being windingly coupled around the spool and having a cord distal end extending through the dispense aperture of the lid, the retraction cord absorbing, or being coated with, the gel solution, the retraction cord being dissolvable after installation within a person's gum tissue.

2. The dissolving gingival retraction cord apparatus of claim 1 further comprising the gel solution comprising at least 20% ferric sulfate.

3. The dissolving gingival retraction cord apparatus of claim 1 further comprising a cap coupled to the lid, the cap being hingingly coupled to the lid sidewall to selectively cover or expose the lid top side.

4. The dissolving gingival retraction cord apparatus of claim 3 further comprising the lid top side having a recessed perimeter portion; a rim of the cap selectively engaging the recessed perimeter portion of the lid top side.

5. The dissolving gingival retraction cord apparatus of claim 1 further comprising the spool including a spool shaft and a spool end cap.

6. The dissolving gingival retraction cord apparatus of claim 1 further comprising a cutter coupled to the lid, the cutter being coupled to the lid top side to cut the retraction cord.

7. The dissolving gingival retraction cord apparatus of claim 6 further comprising the cutter having a base portion and a lip portion.

8. The dissolving gingival retraction cord apparatus of claim 1 further comprising the container body being cylindrical.

9. A dissolving gingival retraction cord apparatus comprising:
a container body having a container bottom side, a container sidewall, and an open container top side defining a container inside, the container body being cylindrical;
a gel solution coupled within the container body, the gel solution comprising at least 20% ferric sulfate;
a lid coupled to the container body, the lid having a lid sidewall selectively engageable with the open container top side and a lid top side, the lid top side having a dispense aperture extending from an outer face through an inner face thereof, the lid top side having a recessed perimeter portion;
a cap coupled to the lid, the cap being hingingly coupled to the lid sidewall to selectively cover or expose the lid top side, a rim of the cap selectively engaging the recessed perimeter portion of the lid top side;
a spool coupled to the lid, the spool being coupled to the inner face of the lid top side and being submerged within the gel solution when the lid is engaged with the container body, the spool including a spool shaft and a spool end cap;
a retraction cord coupled to the spool, the retraction cord being windingly coupled around the spool and having a cord distal end extending through the dispense aperture of the lid, the retraction cord absorbing, or being coated with, the gel solution, the retraction cord being dissolvable after installation within a person's gum tissue; and
a cutter coupled to the lid, the cutter being coupled to the lid top side to cut the retraction cord, the cutter having a base portion and a lip portion.

10. A method of installing a crown comprising:
having a dissolving gingival retraction cord apparatus, the dissolving gingival retraction cord apparatus comprising:
a container body having a container bottom side, a container sidewall, and an open container top side defining a container inside;
a gel solution coupled within the container body;
a lid coupled to the container body, the lid having a lid sidewall selectively engageable with the open container top side and a lid top side, the lid top side having a dispense aperture extending from an outer face through an inner face thereof;
a spool coupled to the lid, the spool being coupled to the inner face of the lid top side and being submerged within the gel solution when the lid is engaged with the container body; and
a retraction cord coupled to the spool, the retraction cord being windingly coupled around the spool and having a cord distal end extending through the dispense aperture of the lid, the retraction cord absorbing, or being coated with, the gel solution, the retraction cord being dissolvable after installation within a person's gum tissue;
cutting a segment of the retraction cord;
inserting the segment around a tooth into the gingival sulcus;
coupling a crown onto the tooth; and
allowing the segment to dissolve.
